(19) United States
(12) Patent Application Publication
(10) Pub. No.: US 2003/0087272 A1
(43) Pub. Date: May 8, 2003

(54) Gene testing method

(75) Inventors: Hideki Kambara, Hachioji (JP); Guohua Zhou, Nanjing (CN); Keiichi Nagai, Higashiyamato (JP); Kazunori Okano, Shiki (JP)

Correspondence Address: Stanley P. Fisher, Reed Smith LLP, Suite 1400, 3110 Fairview Park Drive, Falls Church, VA, 22042-4503, US

(73) Assignee: Hitachi, Ltd.

(21) Appl. No.: 10/179,208

(22) Filed: Jun. 26, 2002

(30) Foreign Application Priority Data
Nov. 6, 2001 (JP) 2001-340133

(51) Int. Cl. C12Q 1/68; C12P 19/34

(52) U.S. Cl. 435/6; 435/91.2

(57) ABSTRACT

A method tests a gene and includes the steps of amplifying a target region to be analyzed of a double-stranded DNA using primers and dNTPs, enzymatically decomposing pyrophosphate formed in the former step and the primers and dNTP used therein, repeatedly synthesizing a complementary strand using primers specific to the sequence and mutation of the target region to be analyzed, and converting a pyrophosphate formed in the step of synthesizing into ATP, allowing the ATP to react with a chemiluminescent reagent to generate chemiluminescence, and monitoring the generated chemiluminescence to thereby detect the presence of the target to be analyzed. This method can easily and simply be performed with high sensitivity at low cost, in which a series of reactions can be performed in a homogenous measuring system.

### FIELD OF THE INVENTION

The present invention relates to gene testing, mutation testing in the gene, and other techniques for testing and detecting DNAs and techniques for genetic diagnoses.

### BACKGROUND OF THE INVENTION

After the completion of reading of the human genome sequence, attempts are now positively made to make full use of gene information in the field of diagnosis and other medical technology. Gel electrophoresis techniques are used predominantly in analyses of DNAs. Among them, capillary array gel electrophoretic systems are widely used as high performance and high throughput DNA analysis systems and are also used in genome analyses.

Subsequent to the genome sequence reading, gene expression profiling and single nucleotide polymorphisms (SNPs) typing in a gene have received attention. The functions of a gene and the relationship between a gene and a disease or drug sensitivity have been investigated by researching genes expressing under different conditions or genetic mutation of individuals. On the basis of accumulated knowledge on the genes, patients are diagnosed with some diseases.

In contrast to the analyses of unknown genes, known genes or the presence of a mutation thereof are to be analyzed upon diagnoses of diseases, and demands have been made on analysis techniques in medical diagnoses that can be performed at low cost. Recently, the analysis of plural genes has increased in importance in addition to diagnoses of diseases each caused by a single gene. The analysis of plural genes is used in diagnoses of diseases caused by different genes in combination with the environment or in the determination of sensitivity to drugs relating to plural genes. This technique has to analyze not a single gene or mutation but plural genes or mutations at once and thereby requires a system which can analyze SNPs and other targets at low cost. The system also includes an amplification process of target regions of genes to be analyzed,

Demands have therefore been made on simple systems other than capillary gel electrophoresis used in the genome analysis. Invader assay, Taqman assay, SSCP (single strand conformation polymorphisms), DNA chip process, and pyrosequencing have been reported as systems that can be used in SNPs analysis and gene probe testing.

More specifically, In the Taqman assay, fluorescence increases with the decomposition of a marker probe upon PCR amplification, and the increase in fluorescence is detected (Reference 1; Proc. Natl. Acad. Sci. USA 88, 7276-7280 (1991)). In the Invader assay, a fluorescent marker probe is decomposed by a combination of the formation of a triple-stranded DNA and the use of an enzyme that recognizes mismatching (Reference 2: Nature Biotech. 17, 292-296 (1999)). In SSCP, a DNA strand including a mutation region is separated on a gel and is detected based on differences in electrophoretic speed, since the DNA strand including a mutation region differs in electrophoretic speed from one containing no mutation (Reference 3: Genomics 5, 874-879 (1989)). In the DNA chip process, a DNA probe array including DNA probes two-dimensionally immobilized as an array is used as a probe (Reference 4: Genomics Research 10, 853-860 (2000)). In addition, a process has been reported in which DNA probes are immobilized to color-coated particles, and these particles are collected and are used as a probe array (Reference 5: Science 287, 451-452 (2000)). These systems use fluorescent markers to detect fluorescence induced by laser excitation and each comprise an excitation laser source and an optical detection system.

The pyrosequencing technique uses stepwise complementary strand synthesis and chemiluminescence and comprises a system for sequentially injecting a trace amount of nucleotide substrate and an optical detection system (Reference 6: Science 281, 363 (1998), Analytical Biochem. 280, 103-110 (2000)).

However, any of these techniques does not satisfy all the fundamental requirements such as low running cost, simple and easy testing that can be performed in a short time, and high reliability, and demands have been made on novel techniques that satisfy all the requirements.

In genetic diagnoses, a demand has been made on a “homogenous” testing technique that is simple and trouble-free and requires only one tube containing a reaction mixture for performing all the procedures. As a possible candidate for this, the present inventors have proposed a DNA mutation typing method utilizing chemiluminescence which can simply and easily be performed at low cost [bioluminometric assay with modified primer extension reaction (BAMPER) (Reference 7: Nucl. Acid Res. 29, e93 (2001)] and have obtained good results. However, even this method requires complicated process steps. For example, the method requires, prior to measurement, the steps of increasing the copy number of a target DNA by means of, for example, polymerase chain reaction (PCR) and purifying the resulting single-stranded DNAs using magnetic particles. The method further comprises the steps of synthesizing a complementary strand specific to a base mutation with the use of the single-stranded DNA as a temperate, converting formed pyrophosphate into ATP, allowing the ATP to react with luciferin and determining the resulting chemiluminescence. In addition, each target requires the preparation and testing of a sample, respectively, and the method thus requires much efforts and time.

As thus described, diagnosis techniques for use in practice must be performed simply and easily, must not require expensive systems, must comprise simple process steps and must be able to analyze plural regions to be analyzed at once.

However, most of conventional techniques developed or used require increasing in the copy number or preparation of a sample on each target prior to analysis and thereby require much efforts, time and costs when a target contains plural regions to be analyzed. The techniques using fluorescence detection require fluorescent marker nucleotides or probe reagents and testing systems including laser, and these reagents and systems are expensive.

### SUMMARY OF THE INVENTION

Accordingly, an object of the present invention is to provide a method for DNA chemiluminescence testing of nucleotide sequences based on a chemiluminescence testing technique which can be performed using a low-cost system and test reagent, which method includes a step of efficiently preparing a sample having multiple regions to be analyzed at low cost, as well as to provide a gene testing technique which can simply and easily be performed with high sensitivity at low cost.

To achieve the above objects, the present invention relates to improvement in a gene testing method including the steps of subjecting a target as a template to a complementary strand synthesis reaction to yield a large amount of pyrophosphate, converting the pyrophosphate into ATP, subjecting the ATP to a chemiluminescent reaction, and detecting the resulting chemiluminescence. The invention proposes a gene testing method in which a large amount of pyrophosphate is obtained directly from a double-stranded DNA without preparation of a single-stranded DNA as a template to thereby improve detection sensitivity. Specifically, the method enables synthesis of a complementary strand specific to a target sequence and mutation directly from a double-stranded DNA and detects the resulting chemiluminescence. When the double-stranded DNA is prepared by PCR, the method further includes the step of decomposing pyrophosphate formed in the aforementioned step and single-stranded DNAs which may adversely affect subsequent reactions, prior to the process steps of synthesizing specific complementary strands and of generating chemiluminescence.

Alternatively, the method may include a chemiluminescence process using pyrophosphate produced in RNA synthesis instead of DNA synthesis.

In any case, the method synthesizes a large amount of a complementary strand from one template DNA to thereby yield pyrophosphate with high efficiency and detects chemiluminescence generated as a result of the subsequent chemiluminescent process step. In addition, the method can prepare plural samples at once even when there are multiple targets to be analyzed, performs chemical reactions in individual reaction subcells respectively and can efficiently detect respective chemiluminescence.

Specifically, the present invention provides, in a first aspect, a gene testing method including the steps of enzymatically synthesizing a DNA strand or RNA strand repeatedly, which DNA strand or the RNA strand is complementary to a sequence of a target region to be analyzed of a single-stranded or double-stranded DNA as a target, converting a pyrophosphate being formed in the former step into ATP, allowing the ATP to react with a chemiluminescent reagent such as luciferin to generate chemiluminescence, and monitoring the generated chemiluminescence to thereby detect the presence of the target sequence.

The gene testing method may be used for detecting the presence of a mutation region in a base sequence in the target and may include the steps of allowing a DNA probe to hybridize with the target, which DNA probe is specific to the sequence of the target region to be analyzed and has an 3′-end capable of hybridizing with a base of the mutation region, if any, of the base sequence, repeating a reaction for synthesizing a complementary strand, converting a pyrophosphate formed in the former step into ATP, allowing the ATP to react with a chemiluminescent reagent such as luciferin to generate chemiluminescence, and monitoring the generated chemiluminescence to thereby detect the presence of the target sequence to thereby detect the presence of the target sequence and the presence of the mutation region.

The gene testing method may include the steps of synthesizing a testing DNA strand having a promoter region specific to a base mutation, synthesizing an RNA using the DNA strand as a template, converting pyrophosphate formed in the step of synthesizing the RNA into ATP, allowing the ATP to react with a chemiluminescent reagent such as luciferin, and monitoring the resulting chemiluminescence to thereby detect the presence of mutation.

The gene testing method may include the steps of preparing a testing DNA strand having a sequence of the target DNA and a promoter sequence, synthesizing an RNA strand using the testing DNA strand as a template to thereby yield the pyrophosphate, converting pyrophosphate formed in the step of synthesizing the RNA into ATP, allowing the ATP to react with a chemiluminescent reagent such as luciferin, and monitoring the resulting chemiluminescence.

In the gene testing method just mentioned above, the step of preparing the testing DNA strand from the target DNA may include the step of controlling the synthesis of the DNA strand depending on whether or not a mutation of the target base sequence to be analyzed is present.

The gene testing method may include the steps of simultaneously synthesizing respective types of DNA strands including respective target regions to be analyzed of the target at once, and performing complementary strand synthesis reactions in partitioned regions or reaction subcells of a reactor, the respective reactions being specific to the respective target regions to be analyzed, converting the resulting pyrophosphate to ATP, and allowing the ATP to react with the chemiluminescent reagent to thereby generate chemiluminescence.

The gene testing method preferably further includes the step of decomposing pyrophosphate, nucleotide substrates, DNA oligomers and other components which may deteriorate the step of detecting chemiluminescence in a liquid containing the testing template DNA, prior to the step of synthesizing the complementary strand for producing pyrophosphate for use in chemiluminescence.

The present invention provides, in a second aspect, a method for testing single nucleotide polymorphisms (SNPs). The method includes the step of controlling the synthesis of a complementary strand of a DNA strand depending on the type of a target base mutation, the DNA strand having a promoter sequence.

The method for testing SNPs may further include the steps of synthesizing an RNA from the DNA strand having a promoter sequence, generating chemiluminescence using pyrophosphate formed during the RNA synthesis, and optically detecting the chemiluminescence.

The present invention provides, in a third aspect, a gene testing method including the steps of amplifying a target region to be analyzed of a double-stranded DNA using primers and dNTPs, enzymatically decomposing pyrophosphate formed in the amplification step, the primers and dNTPs used in the step of amplifying, repeatedly synthesizing a complementary strand using a primer specific to the sequence and mutation of the target region to be analyzed, and converting a pyrophosphate formed in the step of synthesizing into ATP, allowing the ATP to react with a chemiluminescent reagent such as luciferin to generate chemiluminescence, and monitoring the generated chemiluminescence to thereby detect the presence of the target to be analyzed.

The gene testing methods and the SNPs testing method of the present invention relate to improvement in a method including the steps of subjecting a target as a template to a complementary strand synthesis reaction to yield a large amount of pyrophosphate, converting the pyrophosphate into ATP, subjecting the ATP to chemiluminescent reaction, and detecting the resulting chemiluminescence. According to the methods of the invention, a large amount of pyrophosphate is obtained directly from a double-stranded DNA without preparation of a single-stranded DNA as a template to thereby improve detection sensitivity. Specifically, the methods can synthesize a complementary strand specific to a target sequence and mutation directly from a double-stranded DNA and detect the resulting chemiluminescence.

The methods of the present invention utilize repeated complementary strand synthesis in combination of, for example, complementary strand synthesis using a primer specific to a mutation and include the steps of amplifying a copy of a DNA fragment having a specific mutation, converting pyrophosphate formed as a result of the complementary strand synthesis into ATP, and subjecting ATP to chemiluminescence. The methods can therefore enable SNPs typing at low cost. By enzymatically removing unnecessary components such as pyrophosphate formed in the former step and primers which may adversely affect subsequent steps using a catabolic enzyme, a series of the procedures can be performed in a homogenous measuring system. In other words, the procedures from sample preparation to measurement can be performed in one tube. Thus, the invention provides a technique that enables measurement or analysis of samples in security without direct handling of genes and other substances which may cause diseases. In addition, the methods of the present invention can prepare plural samples for detection of plural SNPs at once and can thereby significantly reduce testing cost. The gene testing methods can simply and easily be performed with high sensitivity at low cost.

### BRIEF DESCRIPTION OF THE DRAWINGS

FIG. 1 is a diagram showing the principle of a BAMPER process according to First Embodiment of the present invention, utilizing a cyclic complementary strand synthesis reaction and a chemiluminescent reaction.

FIG. 2 is a diagram schematically showing a process according to Second Embodiment of the present invention in which a target is subjected to a selective complementary strand synthesis using a primer specific to a mutation, and pyrophosphate obtained as a result of PCR amplification is used for the detection of chemiluminescence.

FIG. 3 is a diagram schematically showing a method according to Third Embodiment of the present invention in which probes are immobilized to solid phases in individual cells corresponding to individual types of targets, and complementary strands are synthesized therein.

FIG. 4 is a diagram schematically showing a process according to Fourth Embodiment of the present invention in which a DNA strand having a promoter region is synthesized in a specific manner to a mutation, an RNA is synthesized using the DNA strand, and chemiluminescence is detected by using pyrophosphate formed as a result of the RNA synthesis.

FIG. 5 is a diagram showing an example of artificial mismatching probes in the conventional BAMPER process.

FIG. 6 is a graph illustrating an example of measurements in First Embodiment of the present invention.

### DETAILED DESCRIPTION OF THE PREFERRED EMBODIMENTS

The present invention relates to a technique for efficiently detecting gene mutations such as SNPs. This technique uses a primer (or a DNA probe) that is designed so as to have a 3′-end that matches a target mutation region, and the type of the target mutation is determined depending on whether or not the primer undergoes complementary strand synthesis. By repeating synthesis of a complementary strand from the primer, a large amount of pyrophosphate is produced, and the pyrophosphate is converted into ATP, the ATP is allowed to react with luciferin to generate luminescence, and the luminescence is detected to thereby identify the type of the mutation. The technique uses a double-stranded DNA as a temperate DNA in repeated synthesis of complementary strand and thereby avoids time and efforts in preparation of a single-stranded DNA. The template DNA strand is synthesized and amplified prior to the complementary strand synthesis for the detection of luminescence. According to the technique, pyrophosphate formed in these processes and the primers are enzymatically decomposed to thereby simplify the preparation process of samples. These components may adversely affect the subsequent complementary strand synthesis.

A double-stranded DNA in a target region is amplified by using, for example, PCR, the pyrophosphate formed in the amplification reaction, and the primer and dNTP used in the amplification reaction are enzymatically decomposed, and complementary strands are synthesized repeatedly using a primer specific to the sequence and mutation. By this procedure, a gene can easily be tested without conversion of the target to be analyzed into a single strand.

Alternatively, a DNA strand having a promoter sequence is prepared during complementary strand synthesis using a target as a template, and an RNA is synthesized repeatedly using the DNA strand as a template to thereby detect the target mutation or base sequence with high sensitivity.

According to the methods of the present invention, all the process steps can be performed in a reaction tube without purification of an amplified target to be analyzed into a single strand. The methods can therefore easily test the target fully automatically at low cost. In addition, two primers are used to identify the sequence of the target upon amplification of the target region, and a probe for mutation typing is used to detect the mutation and sequence. Accordingly, the methods enable accurate testing.

The present invention will be illustrated in further detail with reference to several examples below and attached drawings, which are not intended to limit the scope of the invention. The gene testing methods of the present invention can be applied to detection of base mutations (SNPs) in DNA in addition to simple detection of the presence of a DNA. They can also be applied to gene expression profiling.

The methods will be illustrated in the following explanation in detail by taking detection of a base mutation (SNP) in DNA as an example.

Initially, gene testing and mutation testing by BAMPER process will be described. The 3′-end of a DNA probe is designed so as to match the position of a mutation, if any, in a target DNA. In general, a complementary strand is synthesized when the 3′-end of a primer used in complementary strand synthesis is complementary to the target and fully hybridizes with the target, but it is not synthesized or hardly synthesized when the 3′-end does not match the target (i.e., the 3′-end of the primer is not complementary to the target).

In other words, the complementary strand synthesis can be controlled or switched depending on whether or not the 3′-end of the primer matches, or is complementary to, the target.

When the type of a base in the vicinity of the 3′-end of the primer is changed to a different type from that complementary to the target, hybridization of the 3′-end of the primer becomes weak. As a result, a complementary strand is synthesized in the same manner as in the original primer when the 3′-end of the primer is complementary to the target, but it cannot be synthesized when the 3′-end of the primer does not match, or is not complementary to, the target, since the 3′-end of the primer is almost fully apart from the target to thereby inhibit complementary strand synthesis.

The BAMPER process includes the steps of synthesizing a complementary strand with the use of a primer having an artificial mismatch in the vicinity of the 3′-end, converting pyrophosphate formed in the former process step into ATP, and subjecting ATP to a chemiluminescent reaction, and the resulting chemiluminescence is detected.

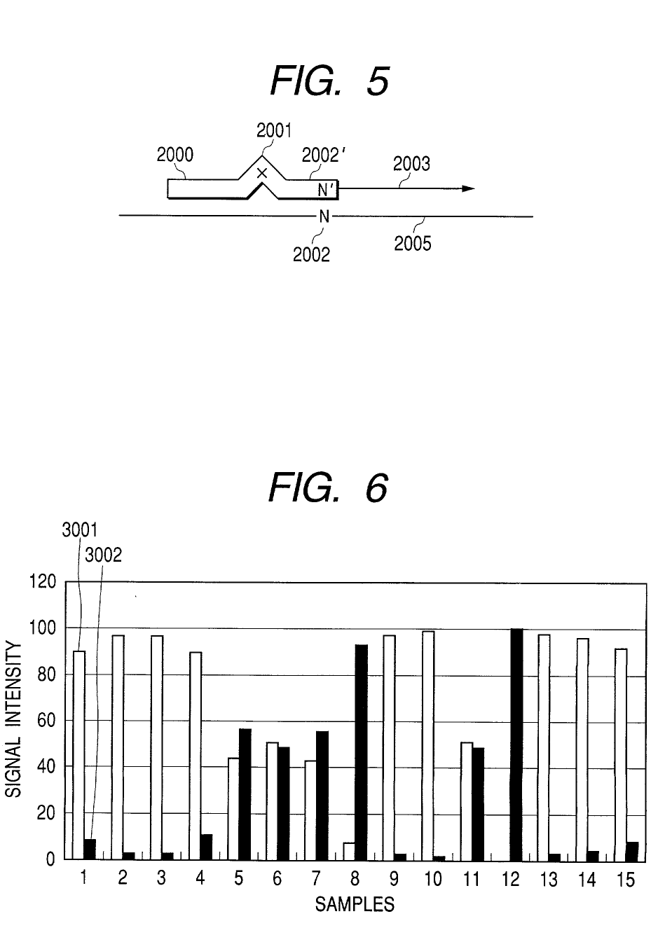

FIG. 5 is a diagram illustrating an example of artificial mismatch probes in the BAMPER process. A primer 2000 hybridizes with a sample DNA 2005. When an 3′-end 2002′ (N′) of the primer 2000 is complementary to a sample DNA region 2002 (N), an extension reaction 2003 occurs. Whether or not the extension reaction occurs depends on whether or not the sample DNA region 2002 is complementary to the primer 2002′. According to this mechanism, SNPs of the region N can be detected. In this procedure, an artificial mismatch is inserted at a position 2001 in the vicinity of the 3′-end of the primer in order to accurately control or switch the occurrence of the extension reaction.

The complementary strand synthesis yields pyrophosphate in an amount corresponding to the length of a synthesized DNA strand, and this technique can yield sensitivity higher than the conventional pyrosequencing by a factor of nearly two orders of magnitude. In the pyrosequencing, a pyrophosphate to be detected is formed corresponding to one base used to extend a strand during complementary strand synthesis.

This technique can also detect the presence of a mutant and determine whether a sample is homozygous or heterozygous. Specifically, in order to distinguish the mutant from the wild accurately, two types of probes each having an end complementary to a mutant or wild are used in reactions, respectively, and the intensities of the resulting luminescence are compared to thereby detect the presence of the mutant and to determine whether the sample is homozygous or heterozygous. Reactions relating to pyrophosphate formation with the complementary strand synthesis and to chemiluminescence will now be schematically illustrated below. The details are described in Reference 6. The symbol X in “-(x)→” means an enzyme X which coexists in the reaction in question.

ssDNA+primer+dNTP-(polymerase)→ssDNA+primer-(dNTP)n+nPPi PPi+APS-(ATP-sulfurylase)→ATP+SO₄²⁻ATP+luciferin+O₂-(luciferase)→AMP+PPi+oxyluciferin+CO₂+hv dNTP-(ATPase)→dNDP+Pi-(ADPase)→dNMP+Pi ATP-(ATPase)→ADP+Pi-(ADPase)→AMP+Pi

First Embodiment

In the present embodiment, a complementary strand is synthesized repeatedly using a primer specific to a target and a double-stranded DNA as a template. The presence of a mutation is detected based on the fact that the type of the resulting pyrophosphate formed during complementary strand synthesis differs depending on the type of a base mutation.

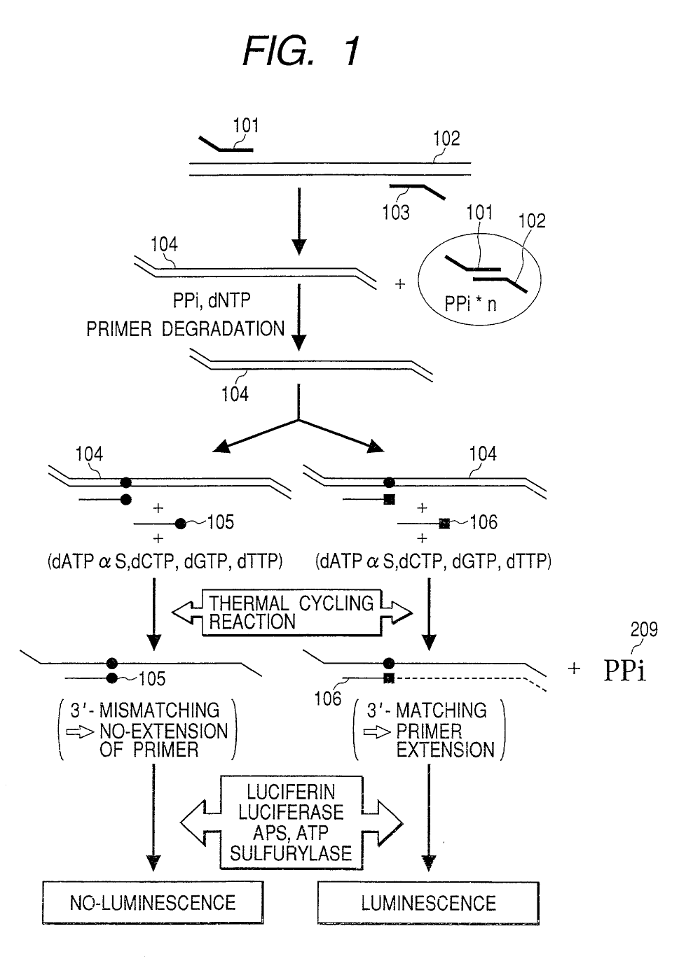

FIG. 1 is a diagram showing the principle of a BAMPER method according to First Embodiment utilizing a cyclic complementary strand synthesis reaction and a chemiluminescent reaction in combination. In First Embodiment, a sample including one target is analyzed, and in Third Embodiment, a sample including plural targets is analyzed. The targets to be analyzed are genome but may be mRNAs. When the target is a mRNA, a cDNA is initially prepared, and the subsequent procedures are performed in the same manner as in genome testing.

A genome DNA is extracted from a specimen to be analyzed, such as the blood, according to a conventional procedure (Reference 8: Molecular Cloning Third Edition (2001) pp 6.8-6.11 (Cold Spring Harbor Laboratory Press)), and a template DNA 102 including a target region is amplified by PCR. In this embodiment, exon 8 of the P 53 gene is amplified using a Human P 53 Exon 4-9 Amplimer Panel (Product Analysis certificate, available from CLONTECH Laboratories, Inc.). The condition for PCR is modified as follows. The PCR amplification requires a first anchored primer 101 (1 pmol), a second anchored primer 103 (1 pmol), DNA polymerase (0.25 u (unit)), and DNA synthesis substrate dNTP (0.2 mM). The genome DNA (5 to 10 ng) is mixed with a solution (total volume: 5 μL) containing these components for the complementary strand synthesis reaction and is subjected to PCR amplification. As thermal cycling in PCR, the sample is heated at 94° C. for 30 seconds, at 55° C. for 30 seconds and at 72° C. for 1 minute, this procedure is repeated a total of 35 times, and the sample is left at 72° C. for 5 minutes.

The first anchored primer 101 has a sequence that hybridizes with one strand of the template DNA 102 at its 3′-end and a sequence that does not hybridizes with the other strand of the template DNA 102 at its 5′-end. The second anchored primer 103 has a sequence that hybridizes one strand of the template DNA 102 at its 3′-end and a sequence that does not hybridizes the other strand of the template DNA 102 at its 5′-end. By this procedure, a testing DNA in an amount of about 0.01 to 0.1 pmol is obtained.

A double-stranded DNA 104 obtained as a result of PCR amplification is the testing DNA. In this technique, chemiluminescence is used for detection, but the resulting solution containing the double-stranded DNA 104 includes components which act as substrates in a chemiluminescent reaction (e.g., dATP and pyrophosphate) and the primers for PCR. The primers react with the template DNA upon the complementary strand synthesis reaction for testing and thereby yield pyrophosphate again. These components adversely affect accurate detection of chemiluminescence generated in a specific complementary strand extension reaction. According to conventional methods, one strand of the primer is labeled with biotin, and a single-stranded DNA alone is extracted by using magnetic particles carrying avidin and is used as a template DNA for testing. However, such magnetic particles are expensive, the template single-stranded DNA is not efficiently purified, and these methods require much efforts and time.

Accordingly, the invented method employs a process in which the substances that will adversely affect chemiluminescence detection are enzymatically decomposed, and the reaction mixture as intact is used in the subsequent process steps. Specifically, the pyrophosphate (PPi) and dNTPs are decomposed with shrimp alkaline phosphatase (0.16 u), and the primers used in PCR are decomposed with exonuclease I (0.32 u) which decomposes single-stranded DNAs. These components are decomposed at 37° C. for 60 minutes. After the decomposition (degradation), the reaction mixture is raised in temperature (80° C. for 10 minutes) to thereby inactivate the enzymes and is subjected to the subsequent complementary strand reaction and chemiluminescent reaction.

After the decomposition of the unnecessary components, each 1 μL of the solution containing the double-stranded DNA 104 is placed in a reactor 1 and a reactor 2, respectively. A DNA probe (1.25 μM) hybridizing a target region to be analyzed of the DNA, dNTPs (each 0.125 mM, containing dATPαS that is resistant to reaction with luciferin instead of dATP), and DNA polymerase (0.1 u) are placed in the reactor 1 and the reactor 2, respectively, and a thermal cycling for DNA complementary strand synthesis reaction (at 94° C. for 10 seconds, at 50° C. for 10 seconds, and at 72° C. for 20 seconds) is repeated five times. The reaction volume is 4 μL.

(SEQ ID NO: 1)

5′-AACAGCTTTGAGGTGCGTGATT-3′

(SEQ ID NO: 2)

5′-AACAGCTTTGAGGTGCGTGATA-3′

A DNA probe 105 having a sequence set forth in SEQ ID NO: 1 and a DNA probe 106 having a sequence set forth in SEQ ID NO: 2 are hybridized with the target to synthesize complementary strands. A complementary strand is extended when the 3′-end of a probe (a complementary strand synthesis primer) is completely complementary to the target, but it is not extended or is hardly extended when the 3′-end of the primer is not completely complementary to the target. Based on this mechanism, the presence of a mutation is detected depending on whether or not the complementary strand synthesis occurs using probes each having an 3′-end corresponding to an expected region of the target mutation. Each of the DNA probes 105 and 106 has an artificial mismatched base at the third base from the 3′-end. The artificial mismatched bases are not complementary to the sequence of the target.

To perform detection accurately, the probe (primer) 105 which is complementary to a wild type of DNA is added to the reactor 1, and the probe (primer) 106 which is complementary to a mutant type of DNA is added to the reactor 2. Subsequently, each 30 μL of a chemiluminescent reagent (0.4 mM luciferin, luciferase, 4 μM APS, 0.2 u/μL ATP sulfurylase) is placed into the reactor 1 and reactor 2, respectively. By this procedure, a pyrophosphate 108 is formed as a result of complementary strand synthesis and is converted into ATP, the ATP is allowed to react with luciferin to generate chemiluminescence, and the resulting chemiluminescence is observed and compared. When the luminescence intensity obtained in the reactor 1 carrying the probe 105 corresponding to the wild type is several times higher than that in the reactor 2 carrying the probe 106 corresponding to the mutant type, the target is the wild type. In a reverse case, the target is the mutant type. When a pair of chromosomes have different types (a wild and a mutant) (heterozygote), the intensities in the two reactors are almost the same. FIG. 6 illustrates an example of actual testing in which P53 gene is subjected to a test, and a specific primer having a sequence set forth in SEQ ID NO: 1 and a specific primer having a sequence set forth in SEQ ID NO: 2 are used. FIG. 6 illustrates the results obtained by using the specific primer having a sequence set forth in SEQ ID NO: 1 as an open bar chart 3001 and the results obtained by using the specific primer having a sequence set forth in SEQ ID NO: 2 as a black bar chart 3002. Specimens 1, 2, 3, 4, 9, 10, 13, 14, and 15 react with the wild type primer 105 alone. Specimens 8 and 12 react with the mutant type primer 106 alone, indicating that they are mutants and are homozygous. Specimens 5, 6, 7, and 11 react with the two primers 105 and 106 to induce an extension reaction, indicating that they are heterozygous. Similar results have been obtained on various genome mutations. In this procedure, a double-stranded DNA is used as the target, but it is also acceptable that the double-stranded DNA is converted into a single strand and is used as the target for complementary strand synthesis.

An ARMS process has been reported in which SNPs typing is performed by using the fact that whether or not 3′-end of a primer matches the target significantly affects complementary strand synthesis (Reference 9: Nucleic Acids Research 17, 2503-2516 (1989)). In this process, a primer for use in mutation typing is used as one of PCR primers, and the resulting products are separated by gel electrophoresis. This process includes the gel electrophoresis step subsequent to PCR amplification and thereby requires much efforts and time. The process uses a fluorescence-labeled primer for fluorescence detection of DNA bands and requires a fluorescence detector, thus inviting high cost.

In addition, the primers used must be carefully selected and the sequences must be minutely designed in order to optimize the intensity of hybridization between the probe (primer) and the target in PCR. If the primer hybridizes with a region other than the target region of genome, unnecessary DNAs are synthesized as a result of PCR. This phenomenon must be avoided.

However, one of PCR primers used in this process must be a primer specific to the mutation, and the primers cannot freely be selected. In some sequences of the target, the mutation cannot be detected according to the process in question. The ARMS process uses dATP and other components in complementary strand synthesis, but these components will adversely affect in chemiluminescence detection since they react with pyrophosphate, although they do not adversely affect in analysis of the resulting DNA strand. A possible solution is, instead of dATP, the use of dATPαS that does not react with chemiluminescence reagents and other components. However, this procedure consumes a large amount of expensive dATPαS.

Additionally, dNTPs are decomposed at elevated temperatures to yield pyrophosphate, and PCR and other processes in which the reaction mixture is exposed to elevated temperatures over and over again are undesired for the chemiluminescent reaction. In contrast, the technique according to First Embodiment can solve these problems. In the technique, primers and dNTP optical to PCR are used to increase the copy number of the target DNA, unnecessary substances for the chemiluminescent reaction are decomposed and removed, and complementary strands are synthesized using a primer specific to the target with a PCR product as a template, followed by the chemiluminescent reaction.

In this technique, the target is a double strand and the specific primer is hybridized with the target at elevated temperatures to thereby synthesize a complementary strand. However, dNTP degradation is trivial, since it takes a short time to elevate the temperature of the sample and the number of elevating the temperature is less than that in PCR. In this connection, complementary strands are synthesized from such a double-stranded DNA at lower efficiency than a single-stranded DNA, and a cycling reaction is suitable for the technique in question.

Second Embodiment

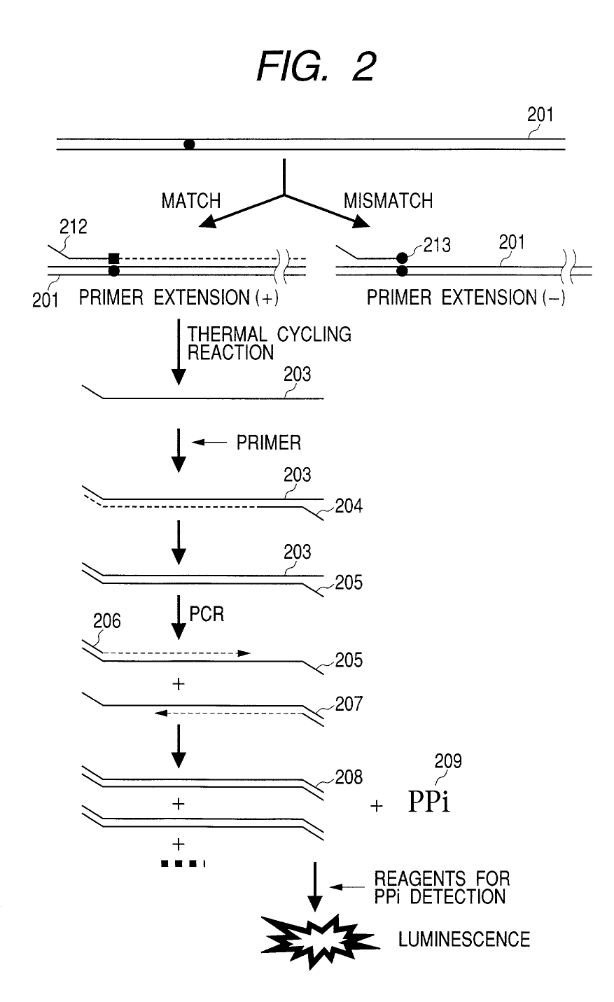

In the present embodiment, complementary strands are synthesized for base mutation typing using two anchored primers each specific to a target sequence, and PCR amplification is performed using a universal primer. FIG. 2 is a diagram schematically showing a method according to Second Embodiment in which a target is subjected to a selective complementary strand synthesis using primers specific to mutations, and pyrophosphate obtained as a result of PCR amplification is used to detect chemiluminescence.

Initially, a genome DNA 201 is extracted in the same manner as in First Embodiment. First anchored primers (first primers) 212 and 213 used herein are each complementary to a target region to be analyzed of the genome DNA 201 and each have an 3′-end designed to correspond to a base mutation region. The first anchored primers 212 and 213 are allowed to hybridize with the genome DNA 201 to thereby synthesize complementary strands (first complementary strand synthesis). The anchor regions of the 5′-ends of the primers 212 and 213 have a universal sequence that does not hybridize with the target region to be analyzed (a common sequence when plural samples are analyzed) and is the same with the sequence of a PCR primer used in a subsequent process step. When the base at the 3′-end of each of the primers 212 and 213 is complementary to the target, a complementary strand is synthesized. If not, the complementary strand is not synthesized. The anchored primer 212 has a base at the 3′-end complementary to the target and thereby proceeds complementary strand synthesis. The anchored primer 213 has a base at the 3′-end not complementary to the target and does not proceed complementary strand synthesis. In other words, the first complementary strand synthesis is a process that recognizes the presence or absence of a mutation. A thermal cycling for DNA complementary strand synthesis reaction is then repeated (first complementary strand synthesis) to thereby yield an extended complementary strand 203 from the primer 212.

Subsequent to the first complementary strand synthesis, unreacted primers 212 and 213, and the genome 201 are removed from the reaction mixture, and the first synthesized DNA strand 203 synthesized by the first complementary strand synthesis is placed in a reactor. Another anchored primer (a second primer) 204 has, at its 5′-end, an anchor region with a sequence that does not hybridize with the first synthesized DNA strand 203. A second complementary strand 205 is then synthesized by a second complementary strand synthesis procedure using the second primer 204 with the first synthesized DNA strand 203 as a template.

A PCR primer 206, a PCR primer 207, dNTPs (including dATPαS instead of dATP) as synthesis substrates, and DNA polymerase are placed in the reactor to perform PCR using the anchor region as a priming region. The PCR primer 206 has the same sequence with that of the anchor region of the 5′-ends of the primers 212 and 213. The PCR primer 207 has the same sequence with that of the anchor region of the 5′-end of the second anchored primer 204. The PCR procedure should preferably be performed at normal temperature, since dNTPs undergo thermal degradation when exposed to elevated temperatures for a long time. To lower the temperature at which DNA strands are separated, it is effective to insert an intercalator. The PCR amplification yields a double-stranded DNA 208 and pyrophosphate 209.

The primers 206 and 207 used in PCR are universal primers which can be selected from primers having a set Tm suitable for PCR and thereby can amplify DNAs stably. If the first complementary strand synthesis does not occur, the PCR product is not produced. Next, a chemiluminescence reagent (a PPi detection reagent including luciferin, luciferase, APS, and ATP-sulfurylase) is placed into the reactor to perform a chemiluminescent reaction. In this reaction, the pyrophosphate 209 formed as a result of PCR is converted into ATP, the ATP is allowed to react with luciferin to generate chemiluminescence, and the chemiluminescence is observed.

The presence or absence of the target mutation can be detected by subjecting two primers having 3′-ends complementary to a wild and a mutant as the first anchored primers 212 and 213 to the above reaction, respectively, and comparing the intensities of the resulting chemiluminescence.

In this procedure, the base mutation is typed by using the first anchored primers 212 and 213, but it is also acceptable that the base mutation is typed by using the second anchored primer 204. The first primers 212 and 213 and the second primer 204 are designed so that they hybridize with the target while sandwiching the region to be analyzed. The second primer 204 is designed so as to have an 3′-end positioning at an expected base mutation region, and the position at which the second primer 204 hybridizes with the target 203 is determined almost uniquely. In contrast, the first primers 212 and 213 hybridize with the target at a position distant from the base mutation region and can thereby be designed so as not to hybridize with other positions of the target 201 by optimizing their Tm and sequences. By performing complementary strand synthesis on an optimized position at first, the base mutation can surely be typed at early stages, thus reducing copying of pseudosequences. From this point, the base mutation typing using the first anchored primers is advantageous.

Next, a complementary strand is synthesized in a specific manner to the mutation using the second primer to thereby yield a PCR template. PCR is then performed using the PCR template and the universal primers, followed by a chemiluminescent reaction. Even when multiple different DNAs are analyzed, the PCR procedure using the universal primers can amplify the multiple DNAs at nearly the same rate respectively and is advantageous. In this case, the plural DNAs are placed in different cells and are subjected to PCR amplification, respectively, and chemiluminescence obtained as a result of the subsequent chemiluminescent reaction is detected on each cell. Comparison of the intensity of the chemiluminescence from each cell detects the presence of the mutation, for example.

Third Embodiment

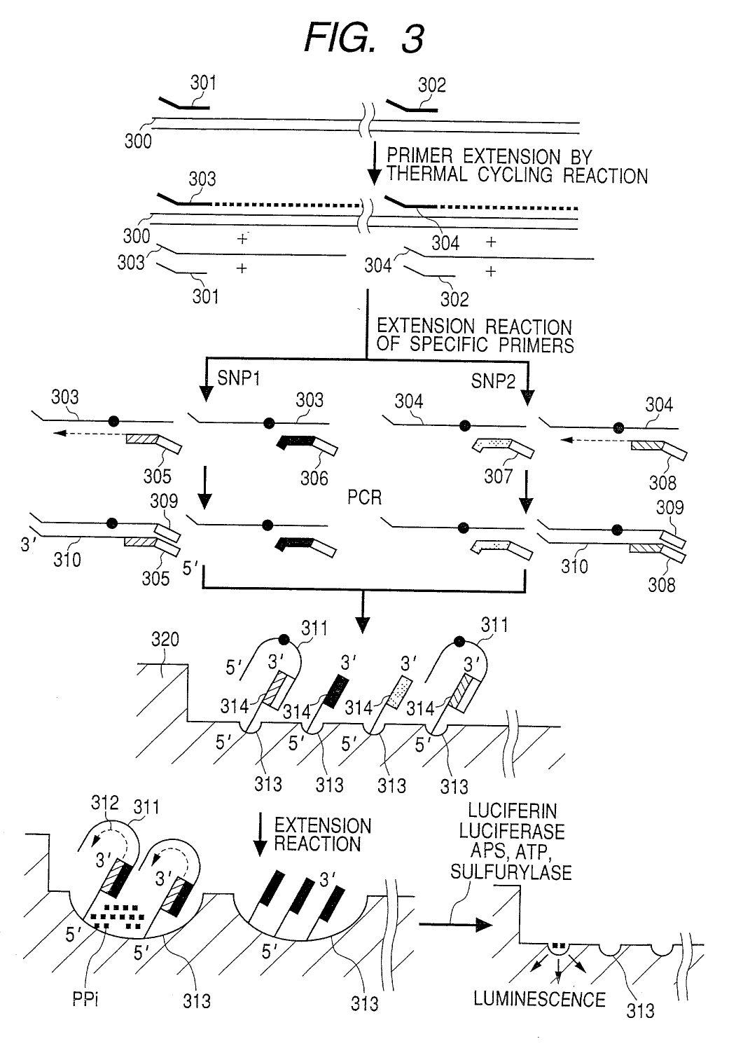

In the present embodiment, plural regions are analyzed. FIG. 3 is a diagram schematically showing a method according to Third Embodiment in which probes are immobilized to solid phases in respective cells corresponding to respective types of targets, and complementary strands are synthesized therein.

In contrast to First Embodiment and Second Embodiment, a DNA strand for complementary strand synthesis is extracted by using a DNA probe immobilized to the surface of a solid phase in the present embodiment, followed by complementary strand synthesis and chemiluminescent reaction. From practical viewpoint, it is preferred that amplification of DNA fragments by, for example, PCR is performed in one reactor, and the complementary strand synthesis reaction for use in chemiluminescence or the complementary strand synthesis reaction for mutation typing is performed in partitioned reaction subcells. However, modifications are also acceptable and will be described below.

In this procedure, at least three complementary strand synthesis process steps are performed, including a first complementary strand synthesis using an anchored primer with a genome as a target, a second complementary strand synthesis using the first complementary strand formed by the first complementary strand synthesis as a template, and a complementary strand synthesis for chemiluminescence using a DNA strand captured by the immobilized probe. One of these complementary strand synthesis process steps must be switched depending on whether or not a mutation is present.

In a first modification, a second probe (a second primer) having an anchor sequence is used in SNPs typing. In the first modification, a complementary strand is synthesized using the second probe, and multiple DNA fragments are amplified by multiple PCR using two common primers having the same sequence as in the anchor region. After the amplification, the multiple different DNA fragments are allowed to hybridize with capture probes immobilized to partitioned subcells in the reactor, respectively, and undergo complementary strand synthesis repeatedly.

Primers for the first complementary strand synthesis using, for example, a genome 300 as a template are anchored primers 301 and 302. The anchored primers 301 and 302 are designed so that complementary strands to be synthesized include the target region to be analyzed. The sequences of the anchor regions at the 5′-ends of the anchored primers 301 and 302 do not hybridize with the genome 300. To detect with high sensitivity, it is also effective to perform the first complementary strand synthesis reaction in a thermal cycling manner to thereby increase the copy number of the complementary strand.

After the first complementary strand synthesis, surplus primers 301 and 302 are removed, and extended complementary DNA strands (first complementary strands) 303 and 304 obtained from the anchored primers 301 and 302 are separated from the genome 300. Second primers are then added to the first complementary strands 303 and 304 to perform the second complementary strand synthesis using the first complementary strands 303 and 304 as templates.

The second primers are anchored primers 305, 306, 307, and 308 that can identify the target sequence, can detect the presence of a mutation and are specific to the target. They are designed so as to have an 3′-end positioning at the mutation region to be analyzed. They each have, as an anchor region, a universal sequence that does not hybridize with the first complementary strands 303 and 304 at the 5′-end, and the universal sequence is used as a priming region for multiple PCR. Each of these primers has a marker sequence that identify the type of the target DNA and the type of mutation between the universal sequence and the complementary sequence specific to the target. The second primers include primers corresponding to the wild and mutant types, respectively. In the subsequent processes, the wild and mutant types are distinguished and are captured by a probe or are selected before subjecting to the complementary strand synthesis. By using these secondary primers, synthesized DNA strands can carry mutation information on one base as a difference in the marker sequence.

After the second complementary strand synthesis, multiple PCR is performed using two universal primers. The two universal primers are a primer having the same sequence as the anchor sequence of the anchored primers 301 and 302, and a primer having the same sequence as the anchor sequence of the anchored primers 305, 306, 307, and 308 and are used for all the DNA fragments in common. The PCR primers are used in common for all the DNA fragments and completely equivalently act upon them to thereby enable uniform amplification. FIG. 3 also shows a common anchor sequence for PCR 309 and an extended DNA strand 310 from the primer.

After the amplification, the used PCR primers, dNTP, and other unnecessary components are decomposed with catabolic enzymes in the same manner as in First Embodiment. Next, the DNA fragments are captured by immobilized probes while distinguishing the amplified DNA strands using the marker sequences.

The immobilized probes capturing the DNA fragments are subjected to complementary strand synthesis to yield pyrophosphate (PPi), the pyrophosphate is subjected to a chemiluminescent reaction using a chemiluminescent reagent (PPi detection reagent including luciferin, luciferase, APS, and ATP-sulfurylase) to thereby detect chemiluminescence.

Alternatively, DNA probes 314 are immobilized to subcells 313 in a reactor 320, respectively, and respective subcells are allowed to capture respective DNA strand targets depending on the marker sequences, as illustrated in FIG. 3. Each of the DNA strands 311 captured by the probes 314 immobilized to the subcells 313 has a specific sequence hybridizing with the probe 314.

A complementary strand is synthesized using the DNA strand 311 captured by the probe 314 as a template with its terminal universal sequence as a priming region. Subsequently, pyrophosphate (PPi) formed as a result of the complementary strand synthesis is used for chemiluminescence analysis. Specifically, the chemiluminescence reagent (PPi detection reagent including luciferin, luciferase, APS, and ATP-sulfurylase) is injected into the reactor 320 to perform a chemiluminescent reaction, and the resulting luminescence is detected. The pyrophosphate, PCR primers, and other components that will adversely affect the chemiluminescent reaction must sufficiently be decomposed. Among the four types of dNTPs, dATPαS is used instead of dATP as a substrate for the complementary strand synthesis in this process, since dATP reacts with luciferin and thereby generates luminescence. Each of the complementary strands is synthesized in each subcell separately to thereby avoid mixing of pyrophosphates formed in different subcells. When the target is a double-stranded DNA, hybridization of the probes must be performed at elevated temperatures.

Alternatively, instead of the use of immobilized probes, different probes are placed in respective subcells, a mixture of double-stranded DNAs is dispensed to the respective subcells, and a cycling reaction is performed between the target DNA and the probe to synthesize a complementary strand to obtain pyrophosphate. It is also acceptable that probes are immobilized to particles (beads), and respective immobilized probes are placed in respective subcells. In any case, the respective subcells are separated from each other upon the cycling reaction, and a complementary strand alone corresponding to the probe placed in each subcell is synthesized. After the cycling reaction, the chemiluminescent reagent is placed in each subcell to perform a chemiluminescent reaction. Accordingly, it is preferred that the operations until the cycling reaction are performed outside an optical measuring device, and the chemiluminescent reaction and the subsequent optical detection are performed in the optical measuring device. The complementary strand synthesis reactions corresponding to plural samples are performed respective partitioned subcells in one reactor, and SNPs can be typed efficiently.

Fourth Embodiment

Examples 1, 2 and 3 each employ a process in which a DNA complementary strand is synthesized using a DNA strand, such as genome or cDNA, as a template for complementary strand synthesis, and the resulting pyrophosphate is used in a chemiluminescent reaction. The process for repeated synthesis of the complementary strand comprises cleavage of double strands of DNA by heating, and primer (probe) hybridization. However, enzymes for used in the chemiluminescent reaction are decomposed at elevated temperatures, and the reagents must be added after the complementary strand synthesis. There are heat-resistant enzymes, but they are expensive.

Accordingly, the present embodiment discloses a technique for yielding a large amount of pyrophosphate without temperature rise. Specifically, in this technique, an RNA strand is synthesized by using a DNA strand as a template, ATP is produced by using pyrophosphate formed in the RNA synthesis, and the ATP is subjected to a chemiluminescent reaction, the resulting luminescence is monitored to thereby detect the presence of the target DNA and the presence of a mutation.

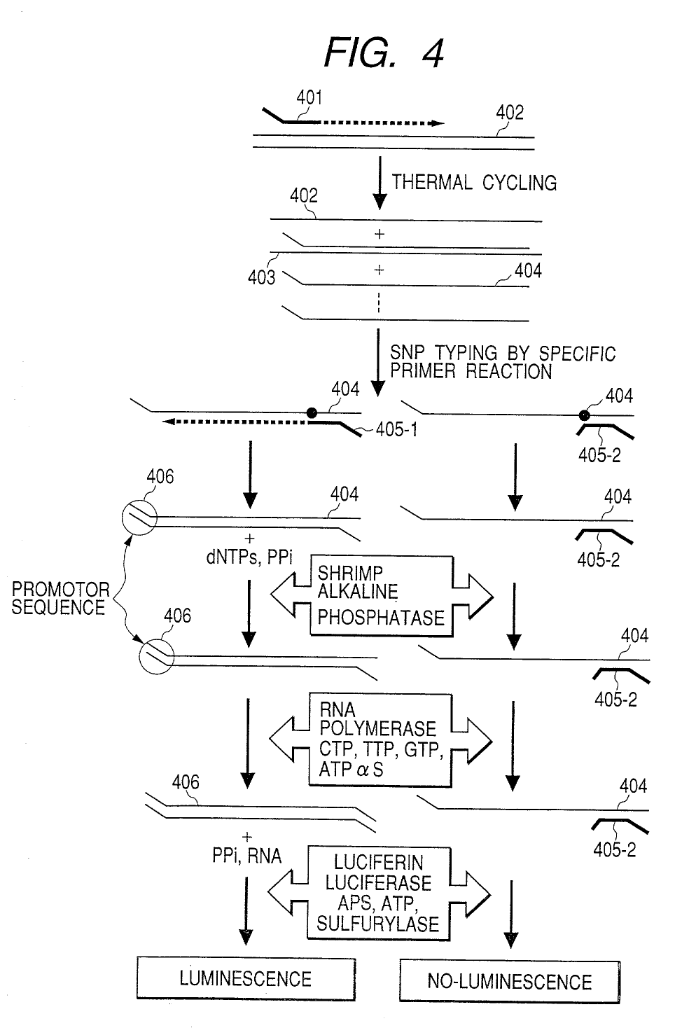

FIG. 4 is a diagram schematically showing a process according to Fourth Embodiment in which a DNA strand having a promoter region is synthesized in a specific manner to mutation, an RNA is synthesized using the DNA strand as a template, and chemiluminescence is detected by using pyrophosphate formed during the RNA synthesis.

To yield an RNA strand with the use of a DNA strand, a promoter sequence is integrated into the DNA strand as the template. In SNPs typing, synthesis of template DNA strands is controlled or switched depending on DNA mutation, and only a template DNA strand corresponding to the target SNP is synthesized. Of course it is also acceptable that RNA complementary strand synthesis is switched depending on the type of SNPs after the synthesis of the DNA strand, but the technique in question will be illustrated by taking control of the synthesis of the template DNA strand for RNA synthesis depending on the type of SNPs as an example.

Initially, a pair of first and second PCR primers is prepared. These PCR primers are designed so as to sandwich a region to be analyzed of a target genome (target DNA) 402. The first primer is an anchored primer 401 having a promoter sequence in an anchor region, and the second primer has bases for SNPs typing at its 3′-end. The 3′-end of the second primer is designed so as to position at the region of the genome to be analyzed on mutation and its sequence is determined by the sequence of the target genome. In contrast, a primer having an optimum sequence selected from regions of 200 base length to 400 base length can be used as the first primer 401. The first primer 401 is first hybridized with the target DNA 402 to thereby synthesize a complementary strand from the first primer 401. Specifically, thermal cycling is performed using the first primer 401 to yield a DNA strand 404, followed by synthesis of a complementary strand of the DNA strand 404 using the second primer. FIG. 4 also shows a DNA strand 403 hybridizing with the primer.

The second primer has an 3′-end positioning at or matching the expected mutation region to be analyzed, and its complementary strand synthesis depends on whether or not the mutation is present. SNPs can be highly precisely typed according to the following procedure. Specifically, two types of primers including a primer that undergoes complementary strand synthesis only in the case that there is the mutation, and a primer that undergoes complementary strand synthesis only in the case that there is no mutation are subjected to complementary strand synthesis independently. Subsequently, the following sequential reactions are performed, and the resulting luminescent intensities are compared.

Multiple SNPs can be simultaneously analyzed by independently performing chemiluminescence in plural reaction subcells that are partitioned and each include the second primer immobilized to a surface of a solid. However, for simplicity sake, Fourth Embodiment will be illustrated below by taking the use of two types of second primers as an example. The two second primers are anchored primers 405-1 and 405-2 each capable of typing gene mutation.

The second primer 405-1 has an 3′-end designed so as to be complementary to a base of the mutation region and undergoes complementary strand synthesis reaction when the target DNA carries the target base mutation. The second primer 405-2 has an 3′-end designed so as not to be complementary to the base in the expected mutation region and does not undergo complementary strand synthesis even when it hybridizes with the target DNA. As a result of these reactions, a DNA strand 406 having a promoter sequence is produced. DNA strands other than the produced double-stranded DNA having the promoter sequence do not contribute to RNA synthesis.

Surplus dNTPs and produced pyrophosphate are then enzymatically decomposed in the same manner as in First Embodiment, since these components will act as a reaction substrate for bioluminescence or chemiluminescence. Specifically, pyrophosphate (PPi) and dNTPs are decomposed with shrimp alkaline phosphatase (0.16 u). In this technique, the presence of single-stranded DNAs and primers is trivial, since the reaction system does not include substrates for complementary strand synthesis.

The enzyme used in decomposition of pyrophosphate and dNTPs is inactivated, and nucleotide substrate NTPs including ATPas instead of ATP, an RNA polymerase, a chemiluminescent reaction substrate, and a chemiluminescent reagent (a PPi detection reagent including luciferin, luciferase, APS, and ATP-sulfurylase) are added to the reaction system to thereby perform RNA strand synthesis and chemiluminescent reaction, in which the pyrophosphate (PPi) formed as a result of RNA strand synthesis is converted into ATP, and the ATP is allowed to react with luciferin, and the resulting luminescence is detected.

As is described above, by subjecting two types of second primers corresponding to wild type and mutant type to reactions, respectively, and comparing the intensities of the chemiluminescence, SNPs can be typed and the frequency of SNPs can be analyzed accurately. By repeating the synthesis of RNA strand at normal temperature, a very large amount of pyrophosphate can be obtained. In addition, the procedure does not require heating, and noise induced by, for example, NTPs degradation can be reduced. This technique can therefore yield high sensitivity.

Alternatively, second primers that can type mutations are immobilized, for example, to the surface of a solid, double-sided DNA having a promoter sequence is synthesized on the solid phase, unnecessary components are removed by washing, and the RNA is synthesized. This type of process using a solid phase is very effective for a sample carrying plural target regions to be analyzed.

Fifth Embodiment

In the present embodiment, the technique of Fourth Embodiment is applied to a sample having plural target regions to be analyzed. In this case, several cycles of a complementary strand extension reaction are performed using a group of first primers each having a promoter sequence and corresponding to different target regions to be analyzed, followed by complementary strand synthesis using a group of second primers each being complementary to the first primer group and having an 3′-end that is designed to hybridize with a mutation region. As the second primer group, respective types of the second primers are immobilized in respective subcells in a reactor.

The second primers are also anchored primers each comprising a site for hybridization with the target, and a linker site for separating the hybridizing site from the surface of the solid. As a result of the complementary strand synthesis, a double-stranded DNA having the promoter region is formed. The targets are hybridizing with the second primers and are thereby immobilized to the subcells. Unnecessary components are then removed by washing, an RNA synthetase, substrates, and a chemiluminescent reagent (a PPi detection reagent comprising luciferin, luciferase, APS, and ATP-sulfurylase) are placed into the subcells to thereby perform RNA synthesis and chemiluminescent reaction in each subcell. Luminescence from each subcell is detected and is compared to thereby type SNPs.

It is also acceptable that dNTPs and pyrophosphate are enzymatically decomposed with a catabolic enzyme in the same manner as in Fourth Embodiment prior to the RNA synthesis. The technique described herein can be applied to simple DNA typing analyses such as detection of the presence of a DNA sequence, in addition to SNPs typing.

While the present invention has been described with reference to what are presently considered to be the preferred embodiments, it is to be understood that the invention is not limited to the disclosed embodiments. On the contrary, the invention is intended to cover various modifications and equivalent arrangements included within the sprit and scope of the appended claims. The scope of the following claims is to be accorded the broadest interpretation so as to encompass all such modifications and equivalent structures and functions.

### SEQUENCE LISTING

<110> HITACHI,LTD.

<120> GENE TESTING METHOD

<130> H01018651A

<150> JAPAN 2001-340133

<151> 2001-11-06

<160> 2

<170> PatentIn version 3.1

<210> 1

<211> 22

<212> DNA

<213> Artificial Sequence

<220>

<223> DNA probe for detecting wild type and having a artificial mismatched base at the third base from the 3′ end.

<400> 1 aacagctttg aggtgcgtga tt 22

<210> 2

<211> 22

<212> DNA

<213> Artificial Sequence

<220>

<223> DNA probe for detecting wild type and having a artificial mismatched base at the third base from the 3′ end.

<400> 2 aacagctttg aggtgcgtga ta 22

What is claimed is:

1. A gene testing method comprising the steps of:
enzymatically synthesizing a DNA strand or RNA strand repeatedly, the DNA strand or the RNA strand being complementary to a sequence of a target region to be analyzed of a single-stranded or double-stranded DNA as a target;
converting a pyrophosphate being formed in the former step into ATP;
allowing the ATP to react with a chemiluminescent reagent to generate chemiluminescence, and
monitoring the generated chemiluminescence to thereby detect the presence of the target to be analyzed.

2. The gene testing method according to claim 1 for detecting the presence of a mutation region in a base sequence in the target, the method further comprising the steps of:
allowing a DNA probe to hybridize with the target, the DNA probe being specific to the sequence of the target region to be analyzed and having an 3′-end capable of hybridizing with a base of the mutation region, if any, of the base sequence; and
repeatedly synthesizing a complementary strand to thereby detect the presence of the mutation region.

3. The gene testing method according to claim 1, further comprising the steps of:
preparing a testing DNA strand having a sequence of the target DNA and a promoter sequence; and
synthesizing an RNA strand using the testing DNA strand as a template to thereby yield the pyrophosphate.

4. The gene testing method according to claim 3 wherein the step of synthesizing a DNA strand having a promoter region specific to a base mutation comprises the step of controlling the synthesis of the DNA strand depending on whether or not a mutation of the target base sequence to be analyzed is present.

5. The gene testing method according to claim 1, comprising the steps of:
simultaneously synthesizing respective types of DNA strands including respective target regions to be analyzed of the target;
performing complementary strand synthesis reactions in partitioned regions or reaction subcells of a reactor, the respective reactions being specific to the respective target regions to be analyzed;
converting the resulting pyrophosphate to ATP; and
allowing the ATP to react with the chemiluminescent reagent to thereby generate chemiluminescence.

6. The gene testing method according to claim 1, further comprising the step of decomposing pyrophosphate, nucleotide substrates, DNA oligomers and other components which may adversely affect the step of detecting chemiluminescence in a liquid containing the testing template DNA, prior to the step of synthesizing the complementary strand for producing pyrophosphate for use in chemiluminescence.

7. A method for testing single nucleotide polymorphisms, the method comprising the step of controlling the synthesis of a complementary strand of a DNA strand depending on the type of a target base mutation, the DNA strand having a promoter sequence.

8. The method for testing single nucleotide polymorphisms, further comprising the steps of:
synthesizing an RNA from the DNA strand having a promoter sequence;
generating chemiluminescence using pyrophosphate formed during the RNA synthesis; and
optically detecting the chemiluminescence.

9. A gene testing method comprising the steps of:
amplifying a target region to be analyzed of a double-stranded DNA using a primer and dNTPs;
enzymatically decomposing pyrophosphate formed in the amplification step, and the primer and dNTPs used in the step of amplifying;
repeatedly synthesizing a complementary strand using a primer specific to the sequence and mutation of the target region to be analyzed;
converting a pyrophosphate formed in the step of synthesizing into ATP;
allowing the ATP to react with a chemiluminescent reagent to generate chemiluminescence; and
monitoring the generated chemiluminescence to thereby detect the presence of the target to be analyzed.
